(19) United States
(12) Patent Application Publication
(10) Pub. No.: US 2025/0235255 A1
(43) Pub. Date: Jul. 24, 2025

(54) DAMAGING CANCEROUS CELLS UTILIZING RADIO FREQUENCY WAVES IN HEATING WITH HEATING ENHANCED BY INFUSION OR INJECTION OF GLUCOSE

(71) Applicant: Howard Rosen, Piedmont (CA)

(72) Inventor: Howard Rosen, Piedmont (CA)

(21) Appl. No.: 18/971,136

(22) Filed: Dec. 6, 2024

Related U.S. Application Data
(63) Continuation of application No. 17/081,347, filed on Oct. 27, 2020, now abandoned.
Application No. 17/081,347 is a continuation of application No. 14/845,804, filed on Sep. 4, 2015, now abandoned.

(51) Int. Cl. A61B 18/12; A61B 18/00; A61B 18/06; A61F 7/00; A61N 1/40; A61N 7/02

(52) U.S. Cl. CPC A61B 18/12; A61B 18/06; A61F 7/007; A61N 1/403; A61N 7/02; A61B 2018/00041; A61B 2018/00577; A61F 2007/0089

(57) ABSTRACT

A method for killing or damaging cancerous cells in a living body comprising introducing glucose or other organic material as a biasing component into heating cancerous cells for purposes of destroying the cancerous cells with a second method of heating, such as by bombardment of energy from an external electromagnetic source, so as to raise the temperature of the tumor or cancerous cells to a degree that they are damaged or expire.

### CROSS-REFERENCE TO RELATED APPLICATIONS

This Application is a Continuation of U.S. patent application Ser. No. 17/081,347, filed Oct. 27, 2029, and which is a Continuation of U.S. patent application Ser. No. 14/845,804, the contents of which are expressly incorporated herein by this reference as though set forth in their entirety and priority to which is claimed.

### BACKGROUND OF THE INVENTION

The present invention relates to treatment of cancer by the killing or damaging of cancer utilizing hyperthermia therapy. The Wikipedia website provides background information on hyperthermia therapy. The following background is edited from information provided on Wikipedia.

Localized and whole-body application of heat has been proposed as a technique for the treatment of malignant tumors. Intense heating will cause denaturation and coagulation of cellular proteins, rapidly killing cells within a tumor. More prolonged moderate heating to temperatures just a few degrees above normal (39.5° C.) can cause more subtle changes. A mild heat treatment combined with other stresses can cause cell death by apoptosis. There are many biochemical consequences to the heat shock response within the cell, including slowed cell division and increased sensitivity to ionizing radiation therapy. The purpose of overheating the tumor cells is to create a lack of oxygen so that the heated cells become over-acidified, which leads to a lack of nutrients in the tumor. This in turn disrupts the metabolism of the cells so that cell death (apoptosis) can set in. In certain cases chemotherapy or radiation that has previously not had any effect can be made effective. Hyperthermia alters the cell walls by means of so-called heat shock proteins. The cancer cells then react very much more effectively to the cytostatic treatments and radiation. If hyperthermia is used conscientiously, it has no serious side effects.

Researchers are also studying substances that help radiation better destroy tumors (radiosensitizers) and those that better protect healthy tissues near the area being treated (radioprotectors). In a further embodiment of the present invention these approaches can be used in conjunction with injection or introduction of glucose (or other organic materials) and radiation to more precisely target and kill cancerous cells while leaving other cells unharmed or less harmed.

There are other techniques by which heat may be delivered. Some of the most common involve the use of focused ultrasound (FUS or HIFU), microwave heating, induction heating, magnetic hyperthermia, and direct application of heat through the use of heated saline pumped through catheters. Experiments with carbon nanotubes that selectively bind to cancer cells have been performed. Lasers are then used that pass light harmlessly through the body, but heat the nanotubes, causing the death of the cancer cells. Similar results have also been achieved with other types of nanoparticles, including gold-coated nanoshells and nanorods that exhibit certain degrees of ‘tunability’ of the absorption properties of the nanoparticles to the wavelength of light for radiation. The success of this approach to cancer treatment rests on the existence of an ‘optical window’ in which biological tissue (i.e., healthy cells) are completely transparent at the wavelength of the laser light, while nanoparticles are highly absorbing at the same wavelength. Such a ‘window’ exists in the so-called near-infrared region of the electromagnetic spectrum. In this way, the laser light can pass through the system without harming healthy tissue, and only diseased cells, where the nanoparticles reside, get hot and are killed.

Magnetic hyperthermia makes use of magnetic nanoparticles, which can be injected into tumors and then generate heat when subjected to an alternating magnetic field. One of the challenges in thermal therapy is delivering the appropriate amount of heat to the correct part of the patient's body. A great deal of current research focuses on precisely positioning heat delivery devices (catheters, microwave, and ultrasound applicators, etc.) using ultrasound or magnetic resonance imaging, as well as of developing new types of nanoparticles that make them particularly efficient absorbers while offering little or no concerns about toxicity to the circulation system. Clinicians also hope to use advanced imaging techniques to monitor heat treatments in real time—heat-induced changes in tissue are sometimes perceptible using these imaging instruments.

One non-invasive cancer treatment involves using radio waves to heat up tiny metals that are implanted in cancerous tissue. Gold nanoparticles or carbon nanotubes are the most likely candidate. Promising preclinical trials have been conducted.

Another method that is entirely non-invasive referred to as Tumor Treating Fields has already reached clinical trial stage in many countries. The concept applies an electric field through a tumor region using electrodes external to the body. Successful trials have shown the process effectiveness to be greater than chemotherapy and there are no side-effects and only negligible time is spent away from normal daily activities. This treatment is still in very early development stages for many types of cancer.

High-intensity focused ultrasound (HIFU) is still in investigatory phases in many places around the world. In China, it has received CFDA approval and over 180 treatment centers have been established in China, Hong Kong, and Korea. HIFU has been successfully used to treat cancer to destroy tumors of the bone, brain, breast, liver, pancreas, rectum, kidney, testes, and prostate. Several thousand patients have been treated with various types of tumors. HIFU has received CE approval for palliative care for bone metastasis. Experimentally, palliative care has been provided for cases of advanced pancreatic cancer.

Hyperthermia therapy in general is a type of medical treatment in which body tissue is exposed to slightly higher temperatures to damage and kill cancer cells or to make cancer cells more sensitive to the effects of radiation and certain anti-cancer drugs. Techniques that may bring local tissues to quite high temperatures, such as radio frequency ablation, are not usually meant by “hyperthermia.” When combined with radiation therapy, it is called thermos-radiotherapy.

Local hyperthermia has shown to be effective when combined with chemotherapy or radiation therapy for cancers such as breast, cervical, prostate, head and neck, melanoma, soft-tissue sarcoma and rectal cancer, among others. Whole-body hyperthermia is generally considered to be a promising experimental cancer treatment, but requires close medical monitoring of the patient, as side effects can be serious.

Hyperthermia is considered the “fourth leg” of cancer treatment. It was historically reserved for the most severe or recurrent cases of cancer. However, there is more evidence to support its use as a primary treatment, as is the practice in parts of Europe, including the Netherlands, Germany, and Austria. Regional hyperthermia is currently becoming more utilized in the United States and elsewhere, as devices come to market. Whole body hyperthermia, using radiant heat chambers is almost always used as an adjuvant therapy. The most effective uses are currently being studied.

Hyperthermia is defined as supra-normal body temperatures. There is no consensus as to what is the safest or most effective target temperature for the whole body. During treatment the body temperature reaches a level between 39.5 and 40.5° C. (103.1 and 104.9° F.). However, other researchers define hyperthermia between 41.8-42° C. (107.2-107.6° F.) (Europe, USA) to near 43-44° C. (109-111° F.). Temperature and time are interrelated, with longer times at temperature meaning more cancer cell kill but also higher risk of toxicity. Hyperthermia may kill or weaken tumor cells, and is controlled to limit effects on healthy cells. Tumor cells, with a disorganized and compact vascular structure, have difficulty dissipating heat. Hyperthermia may therefore cause cancerous cells to undergo apoptosis in direct response to applied heat, while healthy tissues can more easily maintain a normal temperature.

Even if the cancerous cells do not die outright, they may become more susceptible to ionizing radiation therapy or to certain chemotherapy drugs. The heat with applied, local hyperthermia will dilate blood vessels to the tumor, increasing oxygenation of the tumor, thereby making radiation therapy more effective. Oxygen is a potent radio-sensitizer, increasing the effectiveness of a given dose of radiation by forming DNA-damaging free radicals. Tumor cells in a hypoxic environment may be as much as 2 to 3 times more resistant to radiation damage than those in a normal oxygen environment.

Hyperthermia has also been proven to be effective when combined with chemotherapy. Published studies have shown an improvement of 10 year disease free survival in bladder cancer patients treated with combined hyperthermia and chemotherapy with 53% survival, versus those treated with chemotherapy alone, with 15% survival after 10 years.

Intense heating will cause denaturation and coagulation of cellular proteins, rapidly killing cells within a tumor. More prolonged moderate heating to temperatures just a few degrees above normal can cause more subtle changes. A mild heat treatment combined with other stresses can cause cell death by apoptosis. There are many biochemical consequences to the heat shock response within the cell, including slowed cell division and increased sensitivity to ionizing radiation therapy.

Hyperthermia can kill cells directly, but its more important use is in combination with other treatments for cancer. Hyperthermia increases blood flow to the warmed area, perhaps doubling perfusion in tumors, while increasing perfusion in normal tissue by ten times or even more. This enhances the delivery of medications. Hyperthermia also increases oxygen delivery to the area, which may make radiation more likely to damage and kill cells, as well as preventing cells from repairing the damage induced during the radiation session.

Cancerous cells are not inherently more susceptible to the effects of heat. When compared in in vitro studies, normal cells and cancer cells show the same responses to heat. However, the vascular disorganization of a solid tumor results in an unfavorable microenvironment inside tumors. Consequently, the tumor cells are already stressed by low oxygen, higher than normal acid concentrations, and insufficient nutrients, and are thus significantly less able to tolerate the added stress of heat than a healthy cell in normal tissue.

Mild hyperthermia, which provides temperatures equal to that of a naturally high fever, may stimulate natural immunological attacks against the tumor. However it is also induces a natural physiological response called thermo-tolerance, which may tend to protect the treated tumor. Moderate hyperthermia, which heats cells in the range of 40 to 42° C. (104 to 108° F.), damages cells directly, in addition to making the cells radiosensitive and increasing the pore size to improve delivery of large-molecule chemotherapeutic and immunotherapeutic agents (molecular weight greater than 1,000 Daltons), such as monoclonal antibodies and liposome-encapsulateddrugs. Cellular uptake of certain small molecule drugs is also increased. Most local and regional cancer treatments are in this temperature range. Very high temperatures, above 50° C. (122° F.), are used for ablation (direct destruction) of some tumors. This generally involves inserting a metal tube directly into the tumor, and heating the tip until the tissue next to the tube has been killed.

There are many techniques by which heat may be delivered. Some of the most common involve the use of focused ultrasound (FUS or HIFU), infrared sauna, microwave heating, induction heating, magnetic hyperthermia, infusion of warmed liquids, or direct application of heat such as through sitting in a hot room or wrapping a patient in hot blankets.

Local hyperthermia heats a very small area, usually the tumor itself. In some instances, the goal is to kill the tumor by heating it, without damaging anything else. The heat may be created with microwave, radiofrequency, ultrasound energy or using magnetic hyperthermia. Depending on the location of the tumor, the heat may be applied to the surface of the body, inside normal body cavities, or deep in tissue through the use of needles or probes. One relatively common type is radiofrequency ablation of small tumors. This is easiest to achieve when the tumor is on a superficial part of the body, which is called superficial hyperthermia, or when needles or probes are inserted directly into the tumor, which is called interstitial hyperthermia.

Regional hyperthermia heats a larger part of the body, such as an entire organ or limb. Usually, the goal is to weaken cancer cells so that they are more likely to be killed by radiation and chemotherapeutic medications. This may use the same techniques as local hyperthermia treatment, or it may rely on blood perfusion. In blood perfusion, the patient's blood is removed from the body, heated up, and returned to blood vessels that lead directly through the desired body part. Normally, chemotherapy drugs are infused at the same time. One specialized type of this approach is continuous hyperthermic peritoneal perfusion (CHPP), which is used to treat difficult cancers within the peritoneal cavity (the abdomen), including primary peritoneal mesothelioma and stomach cancer. Hot chemotherapy drugs are pumped directly into the peritoneal cavity to kill the cancer cells.

Whole-body hyperthermia heats the entire body to temperatures of about 39 to 43° C. (102 to 109° F.), with some advocating even higher temperatures. It is typically used to treat metastatic cancer, that is, cancer that has spread to many parts of the body. Techniques include infrared hyperthermia domes which include the whole body or the body apart from the head, putting the patient in a very hot room/chamber, or wrapping the patient in hot, wet blankets or a water tubing suit. Others have submerged patients in wax. Methods of pumping the blood outside of the body through heating elements have also been applied. Moderate hyperthermia treatments usually maintain the temperature for about an hour or so.

The schedule for treatments has varied between study centers, as nobody knows what is most effective and treatment schedules have been made based on cell culture or animal studies, or simply been built around a course of planned chemotherapy. After being heated, cells develop resistance to heat, which persists for about three days and reduces the likelihood that they will die from direct cytotoxic effects of the heat. Some even suggest maximum treatment schedule of twice a week. Japanese researchers treated patients with “cycles” up to four times a week apart. Radio-sensitivity may be achieved with hyperthermia, and using heat with every radiation treatment may drive the treatment schedule.

One of the challenges in thermal therapy is delivering the appropriate amount of heat to the correct part of the patient's body. For this technique to be effective, the temperatures must be high enough, and the temperatures must be sustained long enough, to damage or kill the cancer cells. However, if the temperatures are too high, or if they are kept elevated for too long, then serious side effects, including death, can result. The smaller the place that is heated, and the shorter the treatment time, the lower the side effects. Conversely, tumor treated too slowly or at too low a temperature will not achieve therapeutic goals. The human body is a collection of tissues with differing heat capacities, all connected by a dynamic circulatory system with variable relationship to skin or lung surfaces designed to shed heat energy. All methods of inducing higher temperature in the body are countered by the thermo-regulatory mechanisms of the body. The body as a whole relies mostly on simple radiation of energy to the surrounding air from the skin (50% of heat lost this way) which is augmented by convection (blood shunting) and vaporization through sweat and respiration. Regional methods of heating may be more or less difficult based on the anatomic relationships, and tissue components of the particular body part being treated. Measuring temperatures in various parts of the body may be very difficult, and temperatures may locally vary even within a region of the body.

To minimize damage to healthy tissue and other adverse effects, attempts are made to monitor temperatures. The goal is to keep local temperatures in tumor bearing tissue under 44° C. (111° F.) to avoid damage to surrounding tissues. These temperatures have been derived from cell culture and animal studies. The body keeps itself normal human body temperature, near 37.6° C. (99.7° F.). Unless a needle probe can be placed with accuracy in every tumor site amenable to measurement, there is an inherent technical difficulty in how to actually reach whatever a treating center defines as an “adequate” thermal dose. Since there is also no consensus as to what parts of the body need to be monitored (common clinically measured sites are ear drums, oral, skin, rectal, bladder, esophagus, blood probes, or even tissue needles). Clinicians have advocated various combinations for these measurements. These issues complicate the ability of comparing different studies and coming up with a definition of exactly what a thermal dose actually should be for tumor, and what dose is toxic to what tissues in human beings. Clinicians may be able to apply advanced imaging techniques, instead of probes, to monitor heat treatments in real time; heat-induced changes in tissue are sometimes perceptible using these imaging instruments.

There is the further difficulty inherent in the devices delivering energy. Regional devices may not uniformly heat a target area, even without taking into account compensatory mechanisms of the body. A great deal of current research focuses on how one might precisely position heat-delivery devices (catheters, microwave and ultrasound applicators, etc.) using ultrasound or magnetic resonance imaging, as well as developing new types of nanoparticles that can more evenly distribute heat within a target tissue.

The thermo-acoustic (TA) effect refers to the generation of acoustic waves by electromagnetic (EM) irradiation, such as optical or microwave/radio frequency waves. In the past ten years, thermo-acoustic tomography (TAT) using pulsed EM excitation has undergone tremendous growth. Energy deposition inside biological tissue through the absorption of incident EM pulses will create a transient temperature rise. In the thermo-elastic mechanism of acoustic generation, a sound or stress wave is produced as a consequence of the expansion induced by the temperature variation. Thermo-acoustic signals are temperature dependent, which is an ideal characteristic for use in monitoring biological tissue temperature.

By itself, hyperthermia alone has demonstrated the ability to treat cancer. It is known that it significantly increases the effectiveness of other treatments.

When combined with radiation, hyperthermia is particularly effective at increasing the damage to acidic, poorly oxygenated parts of a tumor, and cells that are preparing to divide. Hyperthermia treatment is most effective when provided at the same time, or within an hour, of the radiation.

In the past decade hyperthermia treatments in conjunction with radiation have been used with curative intent in patients with early stage cancers of the breast, head and neck, and prostate. According to peer-reviewed scientific publications, Hyperthermia treatment has shown an improvement of 38% (53% vs. 15%) in Bladder Cancer when combined with Chemotherapy versus chemotherapy alone. In Breast cancer patients, an article by Vernon, et al in 1996 showed an improved response of 18% (59% vs. 41%) when radiation treatment was combined with Hyperthermia, and a 41% response in patients treated with radiation alone. Other cancer types that show a significant clinical response have been: Melanoma and Skin Cancer, Soft Tissue Sarcoma, Bladder, Cervical, Prostate, Rectal, Axilla and Chest Wall as well as recurrent or previously irradiated cancers.

Whole-body hyperthermia has yet to be practically combined with radiation, but it may be useful for chemotherapy and immunotherapy.

A body of research postulates that overheating the tumor cells is meant to create a lack of oxygen so that the heated tumor cells become over acidified, which leads to a lack of nutrients in the tumor. This in turn disrupts the metabolism of the cells so that cell death (apoptosis) can set in. In certain cases chemotherapy or radiation that has previously not had any effect can be made effective. Because hyperthermia alters the cell walls by means of so-called heat shock proteins, cancer cells then react very much more effectively to the cytostatic type treatment and radiation. If hyperthermia is used conscientiously, it has no serious side effects. However whole body hyperthermia or local hyperthermia has better results when associated with infusion therapies (such as Vitamin C, B17, or Oxygen) and can be combined with an insulin potentiation therapy as a way to administrate a low dose chemotherapy.

Research in Russia has shown interesting results with extreme hyperthermia (body temperatures of 43.5 to 44° C. (110.3 to 111.2° F.)) where hyperthermia is used in cancer therapy, HIV therapy and Virus and Immune system illnesses. In a clinical trial, 30 patients received 4 extreme hyperthermia sessions within 70 days and 200 days later. All 30 patients showed fewer viruses) and an increase in CAD4+ (45%), which is 20 times higher when compared with the HAART Therapy.

The application of heat to treat certain conditions, including possible tumors, has a long history. Ancient Greeks, Romans, and Egyptians used heat to treat breast masses; this is still a recommended self-care treatment for breast engorgement. Medical practitioners in ancient India used regional and whole-body hyperthermia as treatments.

During the 19th century, tumor shrinkage after a high fever due to infection had been reported in a small number of cases. Typically, the reports documented the rare regression of a soft tissue sarcoma after erysipelas (an acute streptococcus bacterial infection of the skin; a different presentation of an infection by “flesh-eating bacteria”) was noted. Efforts to deliberately recreate this effect led to the development of Coley's toxin. A sustained high fever after induction of illness was considered critical to treatment success. This treatment is generally considered both less effective than modern treatments and, when it includes live bacteria, inappropriately dangerous.

Around the same period Westermark used localized hyperthermia to produce tumor regression in patients. Encouraging results were also reported by Warren when he treated patients with advanced cancer of various types with a combination of heat, induced with pyrogenic substance, and x-ray therapy. Out of 32 patients, 29 improved for 1 to 6 months.

External, localized hyperthermia treatments sensitize tumors to radiation and chemotherapy, making those treatments more effective. This device uses ultrasound technology to create heat at superficial and deep tumor sites. Hyperthermia may be combined with gene therapy, particularly using the heat shock protein 70 promoter.

Two major technological challenges make hyperthermia therapy complicated: the ability to achieve a uniform temperature in a tumor, and the ability to precisely monitor the temperatures of both the tumor and the surrounding tissue. Advances in devices to deliver uniform levels of the precise amount of heat desired, and devices to measure the total dose of heat received, are hoped for.

In locally advanced adenocarcinoma of middle and lower rectum, regional hyperthermia added to chemo-radiotherapy achieved good results in terms of rate of sphincter sparing surgery.

External-beam radiation therapy is the most common type of radiation treatment, and it involves giving radiation from a machine located outside the body. It can treat large areas of the body, if necessary. The machine typically used to create the radiation beam is called a linear accelerator. Computers and software are used to adjust the size and shape of the beam and to direct it to target the tumor while attempting to avoid radiation of the healthy tissue that surrounds the cancer cells.

### BRIEF SUMMARY OF THE INVENTION

It would be an improvement to the prior art relating to treatment of cancer with hyperthermia therapy if cancer cells could be sensitized such that heating techniques would have more effect on cancer cells than on surrounding or normal tissues.

It is known that all cells, including cancer cells, depend on blood sugar (such as simple glucose or originally sugars such as fructose or sucrose) for energy. Because cancer cells have a different energy metabolism, giving more sugar to cancer cells does not speed their growth, and likewise, depriving cancer cells of sugar does not slow their growth. But glucose is quickly and readily absorbed by cancer cells and tissues that are using more energy-including cancer cells—absorb greater amounts of glucose.

The 1931 Nobel laureate in medicine, German Otto Warburg, Ph.D., first discovered that cancer cells have a fundamentally different energy metabolism compared to healthy cells. The crux of his Nobel thesis was that malignant tumors frequently exhibit an increase in anaerobic glycolysis—a process whereby glucose is used as a fuel by cancer cells with lactic acid as an anaerobic byproduct—compared to normal tissues. Warburg hypothesized that cancer growth is caused by tumor cells mainly generating energy (as e.g. adenosine triphosphate/ATP) by anaerobic breakdown of glucose (known as fermentation, or anaerobic respiration). This is in contrast to healthy cells, which mainly generate energy from oxidative breakdown of pyruvate. Pyruvate is an end product of glycolysis, and is oxidized within the mitochondria. Utilizing more energy implies that cancer cells supplied with more glucose will absorb and burn that glucose at a higher rate than normal cells and therefore will heat up in comparison to normal cells or normal surrounding cells. That is, given injections of glucose, cancer cells will heat themselves to a temperature higher than normal cells or surrounding normal cells also exposed to the glucose.

Supplying further energy to heat both cancer cells and normal cells results in both the normal cells and the cancer cells to be heated above normal body temperatures with the cancer cells heating further above normal temperatures than the surrounding cells due to an infusion of glucose. This results in cancer cells being heated to a higher temperature than surrounding cells which has an increased negative effect on survival of cancer cells or damage to cancer cells in comparison to the survival of or damage to normal or surrounding cells. Thus, cancer cells are essentially targeted for damage/extinction by providing them with glucose (or other organic material) which causes them to heat up, and then also exposing them to further heating in another manner, such as a form of general radiation, or a targeted form of radiation directed primarily at the cancer cells or a specific region or area containing cancer cells so as to kill or damage the cancerous cells without killing or damaging nearby or surrounding cells (or causing less damage to “normal” cells than to cancerous cells).

In the natural sciences, the term diathermy means “electrically induced heat” using high-frequency electromagnetic currents as a form of physical or occupational therapy. Diathermy is commonly used for muscle relaxation. It is also a method of heating tissue electromagnetically or ultrasonically for therapeutic purposes in medicine. Diathermy is used in physical therapy and occupational therapy to deliver moderate heat directly to pathologic lesions in the deeper tissues of the body.

Diathermy, whether achieved using short-wave radio frequency (range 1-100 MHz) or microwave energy (typically 915 MHz or 2.45 GHz), exerts physical effects and elicits a spectrum of physiological responses, the two methods differing mainly in their penetration capability. Different levels of penetration into living tissue can be achieved based upon selection of different frequencies of electromagnetic waves or based upon selection of a plurality of different frequencies of electromagnetic waves.

There are three forms of diathermy typically employed by physical and occupational therapists are ultrasound, short wave and microwave. The application of moderate heat by diathermy increases blood flow and speeds up metabolism and the rate of ion diffusion across cellular membranes.

Ultrasound diathermy employs high-frequency acoustic vibrations which, when propelled through the tissues, are converted into heat. This type of diathermy is especially useful in the delivery of heat to selected musculatures and structures because there is a difference in the sensitivity of various fibers to the acoustic vibrations; some are more absorptive and some are more reflective. For example, in subcutaneous fat, relatively little energy is converted into heat, but in muscle tissues there is a much higher rate of conversion to heat. The therapeutic ultrasound apparatus generates a high-frequency alternating current, which is then converted into acoustic vibrations. The apparatus is moved slowly across the surface of the part being treated. Ultrasound is a very effective agent for the application of heat.

Short wave diathermy machines use two condenser plates that are placed on either side of the body part to be treated. Another mode of application is by induction coils that are pliable and can be molded to fit the part of the body under treatment. As the high-frequency waves travel through the body tissues between the condensers or the coils, they are converted into heat. The degree of heat and depth of penetration depend in part on the absorptive and resistance properties of the tissues that the waves encounter. Short wave diathermy operations typically use the band frequencies of 13.56, 27.12, and 40.68 megahertz. Most commercial machines operate at a frequency of 27.12 MHz, a wavelength of approximately 11 meters.

Microwave diathermy uses microwaves, radio waves which are higher in frequency and shorter in wavelength than the short waves referenced above. Microwaves, which are also used in radar, have a frequency above 300 MHz and a wavelength less than one meter. Most, if not all, of the therapeutic effects of microwave therapy are related to the conversion of energy into heat and its distribution throughout the body tissues. This mode of diathermy is considered to be the easiest to use, but the microwaves typically have a relatively poor depth of penetration. Hyperthermia induced by microwave diathermy can raise the temperature of deep tissues from 41° C. to 45° C. using electromagnetic power.

Microwave diathermy is used in the management of tumors with conventional radiotherapy and chemotherapy. Hyperthermia has been used in oncology for more than 35 years, in addition to radiotherapy, in the management of different tumors. In 1994, hyperthermia has been introduced in several countries of the European Union as a modality for use in physical medicine and sports traumatology. Its use has been successfully extended to physical medicine and sports traumatology in Central and Southern Europe.

Diathermy is also a therapeutic treatment commonly prescribed for joint conditions such as rheumatoid arthritis and osteoarthritis. In diathermy, a high-frequency electric current is delivered via shortwave, microwave, or ultrasound to generate deep heat in body tissues.

Hyperthermia or thermotherapy or thermal therapy or diathermy are exemplary methods for elevating the temperature of a plurality of cells in a body, in specific areas in a body, or directed to specific levels of penetration into a body.

In a first exemplary embodiment of the present invention, a method for cancer treatment includes the steps of: a) injecting glucose into a body or into an area of a body which includes cells that may be cancerous or pre-cancerous; b) utilizing a heating method such as hyperthermia methods and/or diathermy treatment to heat a selected area of cells in the same body in which within that selected area of cells at least some of the cells are suspected or known to be cancerous, pre-cancerous, or considered to be possibly cancerous or pre-cancerous; c) monitoring the temperature of the body or the selected area of cells in the body and continuing application of heat until a level of heating is achieved at which cancerous cells have potential for being damaged or destroyed while other non-cancerous cells remain at a lower temperature and are not destroyed or are less damaged than the cancerous or pre-cancerous cells.

In a second exemplary embodiment of the present invention, a fuel material such as glucose or other organic material is injected into a body or an area of cells in a body known to potentially include a cancerous or pre-cancerous area. The fuel material is known to be consumed/burned by cancerous cells at a rate faster than ordinary cells. An exemplary heating technique in the form of wide spectrum radio waves is applied to an area of the body which includes at least some of the cancerous cells. The wide spectrum radio waves has controlled power at each of a plurality of frequencies such that heating can be directed to a specific depth within the body or to certain types of cells known to be stimulated/heated by specific frequencies of the radio waves. In this manner, cancerous cells can be heated to a level at which they are damaged or killed, while also resulting in less damage and/or less killing of nearby or surrounding non-cancerous cells.

The above description is exemplary in describing one or more illustrated embodiments of the present invention, and alternatives to these methods could be readily determined or designed by one skilled in the art.

### BRIEF DESCRIPTION OF THE SEVERAL VIEWS OF THE DRAWING

The invention will be better understood by means of the following description, given only as an example and in reference to the attached drawing. Other advantages, purposes and characteristics of the present invention will emerge from the following detailed description and with reference to the attached drawings, in which:

FIG. 1 illustrates a first embodiment of the present invention in which heating and infusion of glucose are combined so as to heat cancer cells to a level of temperature at which they are damaged or killed.

FIG. 2 illustrates second and third embodiments of the present invention which also combine heating and the infusion of glucose and are useful in further describing the method of the present invention.

### DETAILED DESCRIPTION OF THE SEVERAL VIEWS OF THE DRAWING

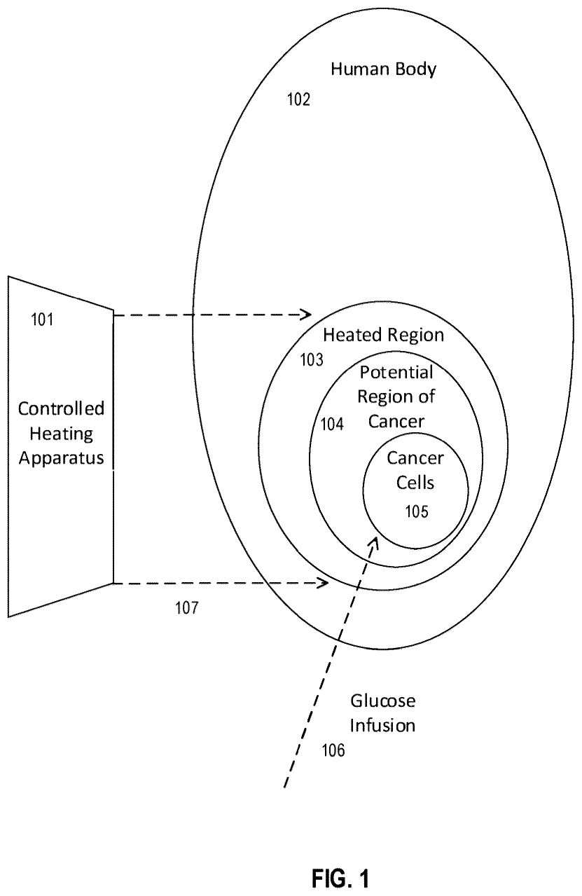

In the first embodiment shown in FIG. 1 used to describe the method of the present invention, a human body 102 is shown which is suspected of having cancer cells within an area 105. The cancerous area 105 is contained within a region 104 identified as a potential region of cancer. The elimination or destruction of cancer cells is performed by injecting or introducing glucose into the potential region 104 of cancer or into a larger region around the potential region of cancer through an IV or through similar apparatus. Glucose may also be introduced by ingestion or injection into a greater area 102 which may include the entire human body. A region 102 to be heated within the human body which surrounds the potential region of cancer is located or identified for heating with known methodologies. A controlled heating apparatus 101 is used to apply heating to the region 102. Such heating raises the temperature of the potential region 104 of cancerous cells above a normal body temperature. The cancerous cells which are included within the region of heating 102 are also raised or increased to a level above the temperature of non-cancerous cells produced by the burning of energy by the cancer cells at a rate of burning which is higher than that of non-cancerous cells as a result of the ready availability and absorption of glucose into the cancerous cells. Thus, this raises the temperature of the cancerous cells to a temperature value at which they are damaged or killed.

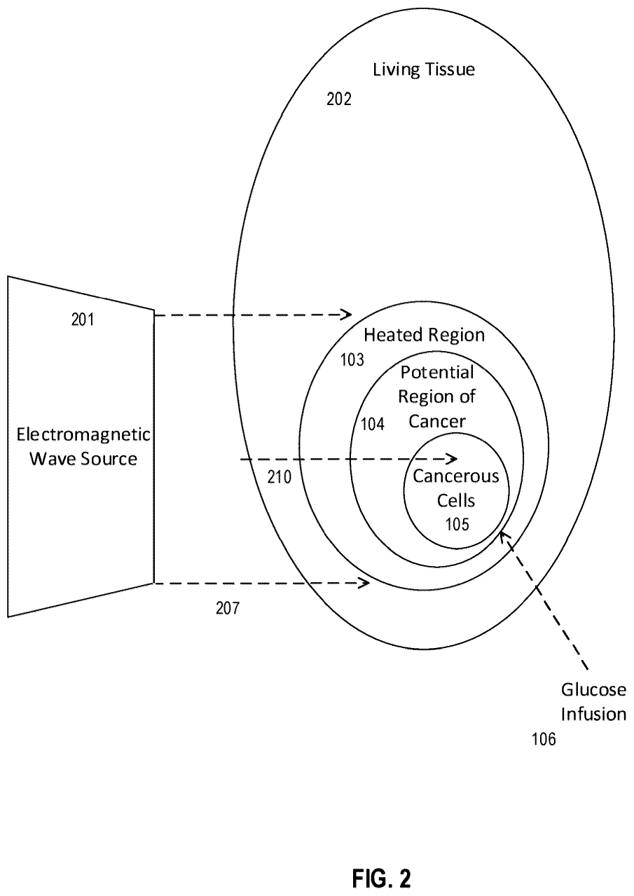

In a second embodiment also shown in FIG. 2 to depict the method of the present invention, an organic agent such as glucose 106 (or other sugars) is introduced via injection or other means into the area of cancerous cells 105 within a body of living tissue 202. The organic compound (e.g. glucose) acts as a temperature biasing component causing an increase in the temperature of the cancerous cells as a result of hyperactive metabolism of certain organic agents in cancerous tissues. As shown in FIG. 2, an external electromagnetic source 201 is then used to bombard an area of living tissue that includes the cancerous cells with electromagnetic energy 207. This causes the cancerous cells to be heated to a temperature that causes destruction or damage of at least a portion of the cancerous cells in area 105. The depth of penetration 210 of the electromagnetic waves is adjusted by selecting a frequency for the electromagnetic waves produced by electromagnetic source 201.

In a third embodiment also shown in FIG. 2, an organic agent such as glucose is introduced via injection or by similar means into the area of cancerous cells 105 which acts as a temperature biasing component thus causing an increase in temperature of the cancerous cells due to hyperactive metabolism of certain organic agents in cancerous tissues. Then, an external electromagnetic source is used bombard an area of living tissue that includes the cancerous cells located within area 105 with electromagnetic energy at a plurality of selected frequencies produced by the external electromagnetic source, thus causing the cancerous cells to be heated to a temperature that causes destruction or damage of at least a portion of the cancerous cells. The frequencies of the plurality of frequencies are optionally selected based upon prior predetermined measurements and studies carried out in a conventional manner to determine a predicted level or depth of penetration 210 into living tissue, and then selecting one or more of a plurality of frequencies to achieve maximum heating in the area or at the depth of penetration 210 known to contain the cancerous cells.

While the principles of the invention have now been made clear and described relative to a number of potential implementations, it will be immediately obvious to those skilled in the art the many modifications or adaptations which can be made without departing from those principles. While the invention has been shown and described with reference to specific illustrated embodiments, it should be understood by those skilled in the art that various changes in form and detail may be made such implementations without departing from the spirit and scope of the teachings of the invention as defined by the following claims.

Having described the illustrated embodiments of the present invention, it will now become apparent to one of skill in the arts that other embodiments or implementations incorporating the teachings of the present invention may be used. Accordingly, these embodiments should not be limited to the disclosed embodiments or implementations but rather should be limited only by the spirit and scope of the following claims.

1. A method for damaging or killing cancer cells in a living human body by damaging or killing cancerous cells comprising the steps of:
heating a first portion of the living human body to a predetermined surrounding temperature, the predetermined surrounding temperature being above normal human body temperature and below that which would kill non-cancerous cells also located in said that-same first portion of the human body; and,
introducing, into cancerous cells in the living human body located in a second portion of the human body that is contained within the first portion of the living human body, via local infusion to the second portion of the human body, an organic material known to be more readily burned by cancerous cells resulting in a plurality of the cancerous cells in the living body burning the organic material with the burning of the organic material raising the temperature of the cancerous cells to a temperature that is above the predetermined surrounding temperature and high enough to damage or kill at least some of the plurality of cancerous cells;
wherein said organic material is glucose;
wherein the step of heating the first portion of the human body is accomplished by directing radio frequency waves into the first portion of the human body, the specific one or more frequencies of the radio waves being selected based upon a desired level of penetration previously determined to be provided by the selected one or more frequencies.
